(19) United States
(12) Patent Application Publication
(10) Pub. No.: US 2025/0275678 A1
(43) Pub. Date: Sep. 4, 2025

(54) Immersive Technology Vision Testing

(71) Applicant: Zenni Optical, Inc., Novato, CA (US)

(72) Inventors: Steven LEE, Barrington, IL (US); Yu Julia ZHEN, Novato, CA (US); ChyrSong TING, Novato, CA (US); Sophia MOH, Daly City, CA (US); Matthew James GOLINO, Brookhaven, GA (US); Justin Paul DEMPSEY, Ottawa (CA); Jeffrey Joseph FILLINGHAM, Dartmouth (CA)

(21) Appl. No.: 19/066,674

(22) Filed: Feb. 28, 2025

Related U.S. Application Data
(60) Provisional application No. 63/560,623, filed on Mar. 1, 2024.

(51) Int. Cl. A61B 3/113; A61B 3/00; A61B 3/11

(52) U.S. Cl. CPC A61B 3/113; A61B 3/005; A61B 3/111

(57) ABSTRACT

The present disclosure relates to methods and systems for implementing vision testing in an extended reality (XR) environment. In some implementations, an XR system includes one or more of: means for displaying optotypes overcoming pixel density limitations of VR displays through algorithmic enhancement; a calibration system for aligning a user's foveal vision with the VR display's central axis using eye-tracking technology; voice control functionality enabling users to navigate and respond within the VR vision testing protocol through spoken commands; a virtual representation of an optometrist placed within the VR environment for guiding the user through the vision test; or a continuous search method implemented for determining visual acuity with precision beyond standard categorization.

### CROSS-REFERENCE TO RELATED APPLICATIONS

This application claims the benefit of U.S. Provisional Application No. 63/560,623, filed Mar. 1, 2024, the entirety of which is incorporated herein by reference.

### TECHNICAL FIELD

The present inventions relate to extended reality systems that can facilitate assessment of visual acuity. More specifically, methods, systems, devices, and non-statutory computer-readable storage media are applied to display optotypes in extended reality environments for assessing visual acuity.

### SUMMARY

In accordance with at least some embodiments disclosed herein is the realization that traditional methods for visual acuity assessment do not allow for dynamic adjustment of test parameters, leading to less accurate assessments, nor can they be implemented to test eyes and vision at home using household devices in a very environment locked manner. Further, in accordance with at least some embodiments disclosed herein is the realization that pixel density limitations of virtual reality (VR) displays can compromise the clarity and effectiveness of optotype presentation. Furthermore, in accordance with at least some embodiments disclosed herein is the realization that existing acuity assessment cannot test high levels of acuity in an extended reality (XR) space due to the limitations of PPD (pixels per degree). The present disclosure addresses these and other deficiencies using novel and innovative solutions that will enable various improvements in vision and health care, as discussed herein.

In accordance with some embodiments, the present disclosure provides a VR system that uses an algorithm to adaptively display optotypes based on real-time user interaction, allowing for more precise visual acuity measurements. In accordance with some embodiments, the system can implement an algorithmic enhancement technique to overcome pixel density limitations, ensuring optotypes are displayed with clarity, thus maintaining the integrity of the vision test.

Some implementations are directed to a new algorithm that allows for a hybrid JND (just noticeable difference) mechanism that does not rely on absolute arclength measurement but rather quantification using visual perception of 2 minor blur states using computerized analyses to differentiate choices.

In some embodiments, using a VR environment (e.g., Quest 3), the system can lock down the variables of an environment like distance, lighting, eye closure, etc. that cannot be done using a pure web-based platform. In other words, in some embodiments, as an optotype is presented on a display that is limited by a pixel resolution, both JND and broad blur comparison are applied to implement visual acuity assessment.

Some implementations apply algorithmic enhancement to overcome pixel density limitations. For example, the system can implement an algorithm that selectively increases contrast and edge definition of optotypes based on the VR display's known pixel density and user's distance from the display.

In some embodiments, the system can dynamically adjust parameters such as sharpness and contrast to optimize visibility. This feature can be implemented through a software module that processes optotype images in real-time, tailoring each image to the specific characteristics of the VR display before it is rendered.

Some implementations apply dynamic adjustment of test parameters. The system assesses user responses in real-time to adaptively modify test parameters, such as optotype size and sequence, enhancing the assessment's accuracy and efficiency. Some implementations apply a feedback loop within the VR application. This feature analyzes response accuracy and timing to adjust subsequent optotypes' difficulty level, ensuring the test remains challenging yet within the user's capabilities.

In some embodiments, the system can use typical optotypes that are shown in regular eye charts. Conversely, in some embodiments, the system uses a combination of both traditional and hybrid images—an example of traditional is the typical E shown in an eyechart; an example of a hybrid image is something that may look like a logo of a company but has pixelation effects that does not require absolutely precision when the image is shrunk to a small size (i.e. it is more about the overall blur effect).

Extended Reality (XR) is an umbrella term encapsulating Augmented Reality (AR), Virtual Reality (VR), Mixed Reality (MR), and everything in between. In this application, any embodiments that apply a VR system can be implemented using an AR or MR system as well.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

For a better understanding of the various described implementations, reference should be made to the Description of Implementations below, in conjunction with the following drawings in which like reference numerals refer to corresponding parts throughout the figures.

FIG. 1 is a visual acuity assessment environment in which an XR system 100 is applied to assess visual acuity for a user, in accordance with some embodiments.

FIG. 2 is a block diagram illustrating a virtual reality, in accordance with some embodiments.

FIG. 3 is a data processing environment having one or more servers communicatively coupled to one or more client devices (e.g., including an XR system), in accordance with some embodiments.

FIG. 4 is a block diagram of an XR headset device configured to implement visual acuity assessment, in accordance with some embodiments.

FIGS. 5 and 6 illustrate the components of an embodiment of a VR vision test system, which can be implements with a software algorithm and hardware integration, to provide a comprehensive view of how the technology is implemented.

For illustrating the overcoming of pixel density limitations through algorithmic enhancement, aspects of the present disclosure can be illustrated with a diagram showcasing the process from optotype selection, through algorithmic enhancement, to display on the VR headset, highlighting the steps taken to adjust for pixel density limitations.

Before and after images can be included as comparative images showing optotypes displayed on VR headsets with or without an algorithmic enhancement, which is optional in some embodiments, thus clearly demonstrating the improvement in clarity and visibility.

### DESCRIPTION OF IMPLEMENTATIONS

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for ease of understanding.

Some embodiments of the present disclosure may include a VR system for vision testing, including means for displaying optotypes overcoming pixel density limitations of VR displays through algorithmic enhancement. Some embodiments may also include a calibration system for aligning a user's foveal vision with the VR display's central axis using eye-tracking technology.

Some embodiments may also include voice control functionality enabling users to navigate and respond within the VR vision testing protocol through spoken commands. Some embodiments may also include a virtual representation of an optometrist placed within the VR environment for guiding the user through the vision test. Some embodiments may also include optotypes including but not limited to tumbling E and tumbling C, with adaptive sizing and orientation based on user performance.

Some embodiments may also include a continuous search method implemented for determining visual acuity with precision beyond standard categorization. For example, instead of jumping at levels of 20/200, 20/100, 20/50, etc. in a standard eye chart; the system can jump to very precise measurements like 20/210 or 20/48. A standard eye chart includes at least one of: Snellen Chart, LogMAR Chart, Tumbling E Chart, Landolt C Chart, ETDRS Chart (Early Treatment Diabetic Retinopathy Study): HOTV Chart, Sloan Letters Chart, LEA Symbols Chart, Jaeger Eye Chart, Kay Pictures, and Bailey-Lovie Chart.

Some embodiments may also include a forced choice method for acuity and sphere determination, offering minimal selection options to refine prescription accuracy. For example, some embodiments of the algorithm disclosed herein can determine if a change in prescription is needed by using acuity and sphere tests (and to see if any increase or decrease in prescription is needed).

Some embodiments may also include an algorithm for optotype selection incorporating a confidence scoring mechanism to verify visual prescription before finalization. A way to display how the finalized results would help a person see better, e.g., by inverting the effects of their uncorrected refractive error thereby showing a simulation on screen that would look clearer and allow them to validate that it does. In some embodiments, the confidence score is determined based on parameters like time taken to answer, and number of incorrect to correct answers; then that is given a score.

In some embodiments, the display has a pixel resolution, and the first set and the second set of optotypes are displayed with a second resolution that is greater than the pixel resolution. Resolution enhance is implemented based on a combination of upscaling, ensuring center location of image so there is maximal resolution, and adjusting for removal of artifacts like antialiasing.

In some embodiments, a user wearing an XR headset will attempt to accommodate, but some embodiments of the algorithm disclosed herein can ensure that there is a relaxation of accommodation due to timing and relaxing of the eyes. The XR system dynamically adjusts the first set of optotypes or the second set of optotypes based on user performance, including enabling an adaptive focus effect. Displaying the first set of optotypes and displaying the second set of optotypes includes applying overlay rendering to simulate physics of ER lenses to reduce distortion in the first set of optotypes and the second set of optotypes.

Some embodiments may also include a method for simulating fixed-distance optotype presentation adjusted according to user's position and viewpoint within the VR environment. For example, in the VR environment, the system can simulate a fixed distance of focal distance (that is not only simulated but actually the true focal distance to the persons eye).

Some embodiments may also include functionality to occlude vision of one eye at a time within the VR headset for isolated eye testing. For example, the VR system adds something to the effect of overcoming software limitations and also implementing a mechanism that comfortably occludes an eye without seeming too harsh and drastic, e.g., using slower occlusion, using gradual transition to occlusion.

In some embodiments, the VR system may include a recorded guide in 2D or 3D format to deliver and guide the vision test within the VR environment. Some embodiments may also include multiple input modalities for user interaction during the test, including but not limited to analog stick, pointer, gaze tracking, hand pointing, and hand gestures. Some embodiments may also include a method for delivering prescriptions directly to users, including association with an online retail account or emailing from the VR environment. Some embodiments may also include a virtual environment for the vision test that simulates an expansive space with audio cues for user guidance and feedback.

In some embodiments, the system may be configured to dynamically adjust the display of optotypes based on an algorithm that accounts for user performance, allowing adaptive focus on specific visual challenges. Some embodiments may also include employ a unique overlay rendering technique that simulates the physics of VR lenses to accurately display optotypes, addressing potential distortions.

In some embodiments, the system utilizes game engine technologies for executing the vision tests, leveraging dynamic content presentation and interactive user experiences. Some embodiments may also include a method for measuring and calibrating interpupillary distance (IPD) within the VR setting to tailor the examination to individual user specifications.

In some embodiments, the VR system includes a mechanism for recalculating visual acuity based on the user's physical distance from optotype displays, thereby adjusting acuity values according to user movement within the VR environment. In some embodiments, the VR system for vision testing is further configured to provide a virtual phoropter within the VR environment, enabling simulation of lens adjustments and refractive error measurements through virtual interaction.

In some embodiments, the adaptive optotype presentation system includes a method for arbitrarily rotating and sizing optotypes to test visual acuity under varied spatial orientations and scales. In some embodiments, the VR system may include a comfortable and immersive testing environment designed to mimic a zen modern office, aimed at relaxing the user to minimize error rates in visual acuity testing.

In some embodiments, the VR system may be further capable of designing and testing new unique optotypes, enabling the exploration of alternative visual acuity and perception testing methods beyond traditional characters and symbols. In some embodiments, the VR system may include a method for determining the level of visual perception of a person in the VR headset without needing to distinguish traditional optotypes, utilizing a series of light-based patterns or gradients that gradually transition in intensity or color, requiring the user to identify changes or thresholds in the visual stimulus, thus assessing visual perception based on sensitivity to contrast or color gradients rather than shape recognition without needing to distinguish traditional optotypes, rather than shape recognition.

In some embodiments, the VR system, additionally configured to simulate various lighting conditions within the VR environment to assess the user's visual acuity and perception under different ambient light settings, mimicking real-world visual scenarios. In some embodiments, the system includes a lens simulation feature that applies color and rendering distortions based on mathematical models of optical physics, to accurately replicate the effects of various lens prescriptions on perceived optotypes.

In some embodiments, the VR system may include a continuous control interface within the VR environment for determining astigmatism correction by adjusting and assessing cylinder power and axis through user interaction. In some embodiments, the system employs a method for dynamically adjusting the perceived distance of optotypes from the user, simulating closer or farther visual tasks without the user physically moving within the VR environment. This simulates closer or farther visual tasks without the user physically moving within the XR environment.

In some embodiments, the VR system, additionally including an audio feedback mechanism that provides verbal cues or sounds in response to user actions during the test, enhancing the interactive experience and guiding the user through the testing process. In some embodiments, the virtual character-guided testing feature offers multiple character options, including realistic human figures, cartoon characters, and abstract forms, to cater to user preferences and enhance engagement.

In some embodiments, the VR system, further configured to incorporate a hand gesture recognition system for interacting with the VR vision test, allowing users to select responses or navigate the testing protocol through natural movements. In some embodiments, the system features multiple redundant user interface modalities for selecting optotypes or responding to tests, ensuring accessibility for users with varying abilities and preferences.

In some embodiments, the VR system, additionally including a method for integrating test results with external health records or vision care provider systems, facilitating the seamless transfer of data for clinical review or further analysis. In some embodiments, the system may be capable of generating a comprehensive report detailing the user's visual acuity, perception levels, and potential corrective lens prescriptions, formatted for easy interpretation by eye care professionals or the user themselves.

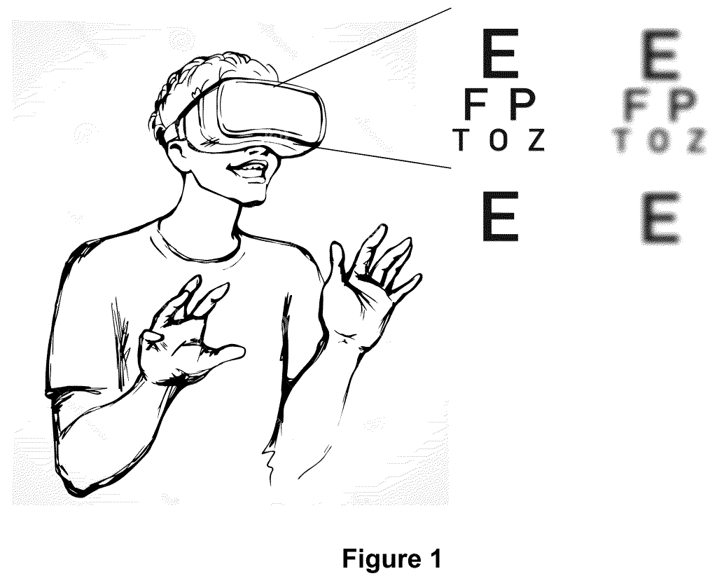

FIG. 1 is a visual acuity assessment environment in which an XR system 100 is applied to assess visual acuity for a user, in accordance with some embodiments.

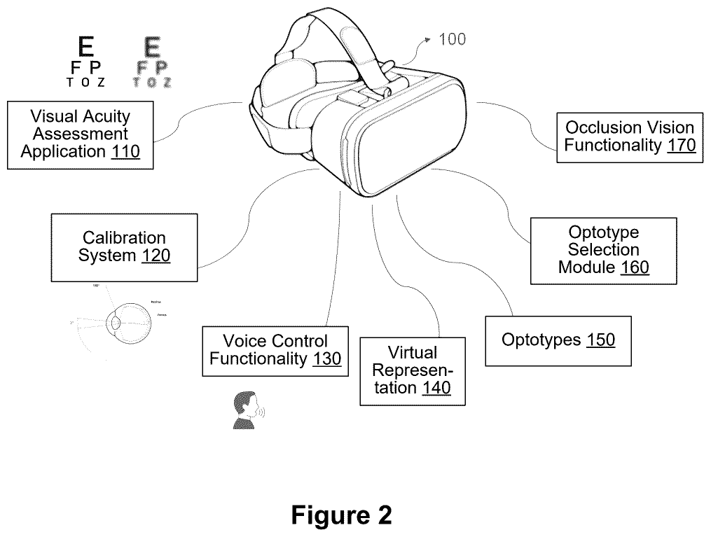

FIG. 2 is a block diagram that describes a virtual reality 100, in accordance with some embodiments. In some embodiments, the virtual reality 100 may include one or more of: means 110 for displaying optotypes overcoming pixel density limitations of VR displays through algorithmic enhancement, a calibration system 120 for aligning a user's foveal vision with the VR display's central axis using eye-tracking technology, voice control functionality 130 enabling users to navigate and respond within the VR vision testing protocol through spoken commands, a virtual representation 140 of an optometrist placed within the VR environment for guiding the user through the vision test, optotypes 150, an algorithm 160 for optotype selection incorporating a confidence scoring mechanism to verify visual prescription before finalization, and functionality 170 to occlude vision of one eye at a time within the VR headset for isolated eye testing.

In some embodiments, the optotypes are selected from a Tumbling E eye chart or a Tumbling C eye chart, and have adaptive sizing and orientation based on user responses. A continuous search method may be implemented for determining visual acuity with precision beyond standard categorization. A forced choice method may be applied for acuity and sphere determination, offering minimal selection options to refine prescription accuracy. A method may be applied for simulating fixed-distance optotype presentation adjusted according to user's position and viewpoint within the VR environment.

In some embodiments, the system may be configured to adjust the display of optotypes dynamically based on an algorithm that accounts for user performance, allowing adaptive focus on specific visual challenges. The system may employ a unique overlay rendering technique that simulates the physics of VR lenses to accurately display optotypes, addressing potential distortions. In some embodiments, the system may utilize. A method for measuring and calibrating interpupillary distance (IPD) within the VR setting to tailor the examination to individual user specifications.

In some embodiments, the VR system may also include a mechanism for recalculating visual acuity based on the user's physical distance from optotype displays, employing an algorithm to adjust acuity values according to user movement within the VR environment. In some embodiments, the VR system is further configured to provide a virtual phoropter within the VR environment, enabling the simulation of lens adjustments and refractive error measurements through virtual interaction.

In some embodiments, a method is applied for arbitrarily rotating and sizing optotypes to test visual acuity under varied spatial orientations and scales. In some embodiments, the system arbitrarily rotates the optotypes and varies the optotype size, and this adaptive optotype presentation system tests visual acuity under varied spatial orientations and scales. In some embodiments, the VR system may also include a comfortable and immersive testing environment designed to mimic a zen modern office, aiming at relaxing the user to minimize error rates in visual acuity testing. In some embodiments, the system may be further capable of. The system may use new unique optotypes that are not used in the standard eye charts and explore alternative visual acuity and perception testing methods beyond traditional characters and symbols. In some situations, the immersive testing environment minimizes error rates in visual acuity testing.

In some embodiments, a method is applied for determining the level of visual perception of a person in the VR headset without needing to distinguish traditional optotypes. The method may utilize a series of light-based patterns or gradients that gradually transition in intensity or color, and require the user to identify changes or thresholds in the visual stimulus. By these means, the system may assess visual perception based on sensitivity to contrast or color gradients rather than shape recognition.

In some embodiments, the system may be configured to simulate various lighting conditions within the VR environment to assess the user's visual acuity and perception under different ambient light settings, mimicking real-world visual scenarios. In some embodiments, the system may also include a lens simulation feature that applies color and rendering distortions based on mathematical models of optical physics, to accurately replicate the effects of various lens prescriptions on perceived optotypes. This accurately replicates the effects of various lens prescriptions on perceived optotypes.

In some embodiments, the VR system may include a continuous control interface within the VR environment for determining astigmatism correction by adjusting and assessing cylinder power and axis through user interaction. In some embodiments, the system may employ a method for dynamically adjusting the perceived distance of optotypes from the user, simulating closer or farther visual tasks without the user physically moving within the VR environment.

In some embodiments, the VR system may also include an audio feedback mechanism that provides verbal cues or sounds in response to user actions during the test, enhancing the interactive experience and guiding the user through the testing process. In some embodiments, the system may employ a method for integrating test results with external health records or vision care provider systems, facilitating the seamless transfer of data for clinical review or further analysis.

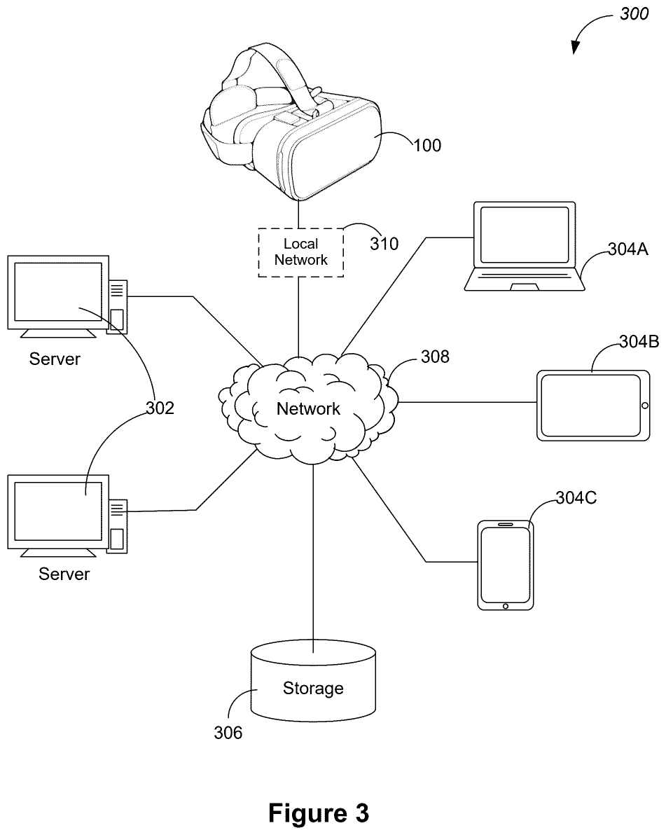

FIG. 3 is an example data processing environment 300 having one or more servers 302 communicatively coupled to one or more client devices 304 (e.g., including an XR system 100), in accordance with some embodiments. The one or more client devices 304 may be, for example, desktop computers 304A, tablet computers 304B, mobile phones 304C, or intelligent, multi-sensing, network-connected home devices (e.g., a depth camera, a visible light camera). In some implementations, the one or more client devices 304 include an XR system 100 (also called a head-mounted display 100) configured to render extended reality content. Each client device 304 can collect data or user inputs, executes user applications, and present outputs on its user interface. The collected data or user inputs can be processed locally at the client device 304 and/or remotely by the server(s) 302. The one or more servers 302 provides system data (e.g., boot files, operating system images, and user applications) to the client devices 304, and in some embodiments, processes the data and user inputs received from the client device(s) 304 when the user applications are executed on the client devices 304. In some embodiments, the data processing environment 300 further includes a storage 306 for storing data related to the servers 302, client devices 304, and applications executed on the client devices 304. For example, storage 306 may store video content, static visual content, and/or audio data.

The one or more servers 302 can enable real-time data communication with the client devices 304 that are remote from each other or from the one or more servers 302. Further, in some embodiments, the one or more servers 302 can implement data processing tasks that cannot be or are preferably not completed locally by the client devices 304. For example, the client devices 304 include a game console (e.g., the XR system 100) that executes an interactive online gaming application. The game console receives a user instruction and sends it to a game server 302 with user data. The game server 302 generates a stream of video data based on the user instruction and user data, and provides the stream of video data for display on the game console and other client devices that are engaged in the same game session with the game console.

The one or more servers 302, one or more client devices 304, and storage 306 are communicatively coupled to each other via one or more communication networks 308, which are the medium used to provide communications links between these devices and computers connected together within the data processing environment 300. The one or more communication networks 308 may include connections, such as wire, wireless communication links, or fiber optic cables. Examples of the one or more communication networks 308 include local area networks (LAN), wide area networks (WAN) such as the Internet, or a combination thereof. The one or more communication networks 308 are, optionally, implemented using any known network protocol, including various wired or wireless protocols, such as Ethernet, Universal Serial Bus (USB), FIREWIRE, Long Term Evolution (LTE), Global System for Mobile Communications (GSM), Enhanced Data GSM Environment (EDGE), code division multiple access (CDMA), time division multiple access (TDMA), Bluetooth, Wi-Fi, voice over Internet Protocol (VOIP), Wi-MAX, or any other suitable communication protocol. A connection to the one or more communication networks 308 may be established either directly (e.g., using 3G/4G connectivity to a wireless carrier), or through a network interface 310 (e.g., a router, switch, gateway, hub, or an intelligent, dedicated whole-home control node), or through any combination thereof. As such, the one or more communication networks 308 can represent the Internet of a worldwide collection of networks and gateways that use the Transmission Control Protocol/Internet Protocol (TCP/IP) suite of protocols to communicate with one another. At the heart of the Internet is a backbone of high-speed data communication lines between major nodes or host computers, consisting of thousands of commercial, governmental, educational and other electronic systems that route data and messages.

In some embodiments, the XR system 100 is communicatively coupled to a data processing environment 300. The XR system 100 includes one or more cameras (e.g., a visible light camera, a depth camera), a microphone, a speaker, one or more inertial sensors (e.g., gyroscope, accelerometer), and a display. In some situations, the camera captures hand gestures of a user wearing the XR system 100. In some situations, the microphone records ambient sound, including user's voice commands.

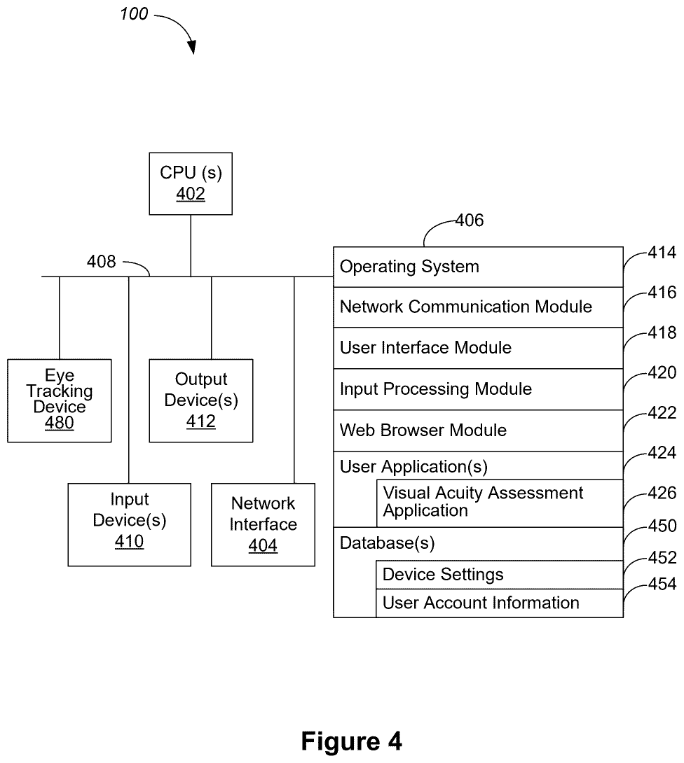

FIG. 4 is a block diagram of an example XR system 100 (e.g., a headset VR device) configured to implement visual acuity assessment, in accordance with some embodiments. The XR system 100, typically, includes one or more processing units (CPUs) 402, one or more network interfaces 404, memory 406, and one or more communication buses 408 for interconnecting these components (sometimes called a chipset). The XR system 100 includes one or more input devices 410 that facilitate user input, such as a keyboard, a mouse, a voice-command input unit or microphone, a touch screen display, a touch-sensitive input pad, a gesture capturing camera, or other input buttons or controls. Furthermore, in some embodiments, the client device 104 of the XR system 100 uses a microphone for voice recognition or an eye tracking device 480 (e.g., a camera) for tracking eyeball movement. In some embodiments, the client device 104 includes one or more optical cameras (e.g., an RGB camera), scanners, or photo sensor units for capturing images. The XR system 100 also includes one or more output devices 412 that enable presentation of user interfaces and display content, including one or more speakers and/or one or more visual displays. In some embodiments, the XR system 100 includes an inertial measurement unit (IMU) integrating sensor data captured by multi-axes inertial sensors to provide estimation of a location and an orientation of the XR system 100 in space. Examples of the one or more inertial sensors of the IMU include, but are not limited to, a gyroscope, an accelerometer, a magnetometer, and an inclinometer.

Memory 406 includes high-speed random access memory, such as DRAM, SRAM, DDR RAM, or other random access solid state memory devices; and, optionally, includes non-volatile memory, such as one or more magnetic disk storage devices, one or more optical disk storage devices, one or more flash memory devices, or one or more other non-volatile solid state storage devices. Memory 406, optionally, includes one or more storage devices remotely located from one or more processing units 402. Memory 406, or alternatively the non-volatile memory within memory 406, includes a non-transitory computer readable storage medium. In some embodiments, memory 406, or the non-transitory computer readable storage medium of memory 406, stores the following programs, modules, and data structures, or a subset or superset thereof:
Operating system 414 including procedures for handling various basic system services and for performing hardware dependent tasks;
Network communication module 416 for connecting each server 102 or client device 104 to other devices (e.g., server 102, client device 104, or storage 106) via one or more network interfaces 404 (wired or wireless) and one or more communication networks 108, such as the Internet, other wide area networks, local area networks, metropolitan area networks, and so on;
User interface module 418 for enabling presentation of information (e.g., a graphical user interface for application(s) 424, widgets, websites and web pages thereof, and/or games, audio and/or video content, text, etc.) at each client device 104 via one or more output devices 412 (e.g., displays, speakers, etc.);
Input processing module 420 for detecting one or more user inputs or interactions from one of the one or more input devices 410 and interpreting the detected input or interaction;
Web browser module 422 for navigating, requesting (e.g., via HTTP), and displaying websites and web pages thereof, including a web interface for logging into a user account associated with a client device 104 or another electronic device, controlling the client or electronic device if associated with the user account, and editing and reviewing settings and data that are associated with the user account;
One or more user applications 424 for execution by the XR system 100 (e.g., games, social network applications, smart home applications, extended reality application, and/or other web or non-web based applications for controlling another electronic device and reviewing data captured by such devices), where in some embodiments, a visual acuity assessment application 426 is executed to display optotypes dynamically on the display of the XR system 100 for assessing visual acuity of a patient; and
One or more databases 450 for storing at least data including one or more of:
   Device settings 452 including common device settings (e.g., service tier, device model, storage capacity, processing capabilities, communication capabilities, etc.) of the XR system 100; and
   User account information 454 for the one or more user applications 424, e.g., user names, security questions, account history data, user preferences, and predefined account settings.

Each of the above identified elements may be stored in one or more of the previously mentioned memory devices, and corresponds to a set of instructions for performing a function described above. The above identified modules or programs (i.e., sets of instructions) need not be implemented as separate software programs, procedures, modules or data structures, and thus various subsets of these modules may be combined or otherwise re-arranged in various embodiments. In some embodiments, memory 406, optionally, stores a subset of the modules and data structures identified above. Furthermore, memory 406, optionally, stores additional modules and data structures not described above.

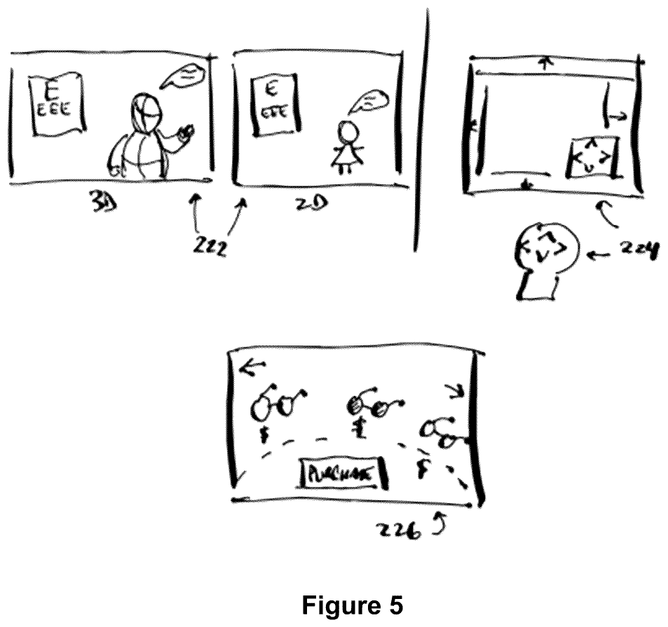

FIG. 5 is a block diagram that further describes the VR system 100 from FIGS. 1-4, in accordance with some embodiments. In some embodiments, the VR system 220 may include one or more of: a recorded guide 222 in 2D or 3D format to deliver and guide the vision test within the VR environment, multiple input modalities 224 for user interaction during the test, association 226 with an online retail account or emailing from the VR environment, and a virtual environment 228 for the vision test that simulates an expansive space with audio cues for user guidance and feedback. The VR system 100 interacts with a user based on a mechanism including, but not limited to, analog stick, pointer, gaze tracking, hand pointing, and hand gestures. This allows users to select responses or navigate the testing protocol through natural movements. The system may also employ a method for requesting a delivery of an eye prescription directly to a user.

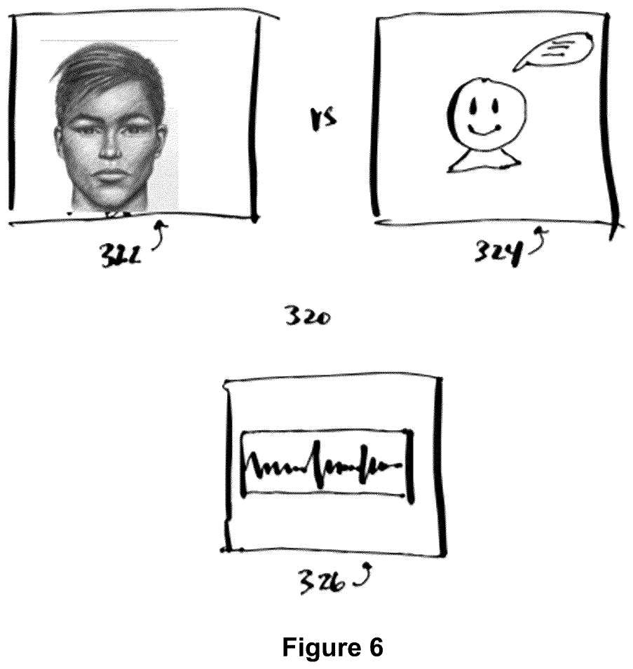

FIG. 6 is a block diagram that further describes the virtual reality 100 from FIG. 1, in accordance with some embodiments. In some embodiments, the virtual character-guided testing feature offers multiple character options 320 including one of realistic human figures 322 and cartoon characters 324. The multiple character options 320 may also include abstract forms 326 configured to cater to user preferences and enhance engagement. In some embodiments, a character is selected based on a user preference for enhancing user engagement.

### Illustration of Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. A method for assessing visual acuity, comprising: at an extended reality (XR) device including one or more processors, memory for storing one or more programs, a display, a microphone, a speaker, and one or more sensors including at least an eye tracking device: identifying a nominal foveal location of an eye using the eye tracking device; setting a central axis of the display based on the nominal foveal location; based on the central axis of the display, displaying a first set of optotypes on the display, the first set of optotypes including one or more first optotypes; obtaining a first user input in response to displaying the first set of optotypes; in response to the first user input, determining a size progression from the first set of optotypes to a second set of optotypes, the first set of optotypes including one or more second optotypes and displaying the second set of optotypes on the display based on the size progression for assessing visual acuity of a user.

Clause 2. The method of Clause 1, wherein the first set of optotypes and the second set of optotypes are displayed concurrently on the display.

Clause 3. The method of Clause 1, wherein the first set of optotypes and the second set of optotypes are displayed sequentially via two successive video clips or two static images on the display, and the size progression is determined and the second set of optotypes are displayed adaptively based on the first user input.

Clause 4. The method of any of Clauses 1-3, wherein the size progression corresponds to a visual acuity assessment resolution that is different from any known visual acuity assessment resolution of a plurality of predefined eye charts.

Clause 5. The method of Clause 4, wherein the second set of optotypes distinct from the predefined eye charts.

Clause 6. The method of any of Clauses 1-5, further comprising: based on an optotype size of the first set of optotypes, an optotype size of the second set of optotypes, the first user input, and the size progression, determining a visual prescription.

Clause 7. The method of Clause 6, further comprising: receiving an optometrist input selecting one of a plurality of accuracy options for the visual prescription, wherein the size progression is determined and the second set of optotypes are presented iteratively, until the selected one of the plurality of accuracy options is satisfied.

Clause 8. The method of any of Clauses 1-7, further comprising: determining a confidence score for determining the size progress of the second set of optotypes; and verifying a visual prescription based on the confidence score.

Clause 9. The method of any of Clauses 1-8, further comprising: wherein the display has a pixel resolution, and the first set and the second set of optotypes are displayed with a second resolution that is greater than the pixel resolution.

Clause 10. The method of any of the preceding Clauses, further comprising: receiving an audio message by the microphone of the XR device; and converting the audio message to the first user input.

Clause 11. The method of any of the preceding Clauses, further comprising: displaying a virtual representation of an optometrist for guiding the user through a vision test.

Clause 12. The method of any of the preceding Clauses, further comprising: adaptively determining a size and an orientation of the second set of optotypes based on the first user input.

Clause 13. The method of any of the preceding Clauses, further comprising: determining the user's position and viewpoint in the XR environment; and enabling a fixed-distance optotype presentation based on the user's position and viewpoint.

Clause 14. The method of any of the preceding Clauses, wherein the display includes a left display and a right display, and the first set and the second set of optotypes are displayed in one of the left display and the right display, the method further comprising: displaying a static blank image in the other one of the left display and the right display to occlude vision of a corresponding eye.

Clause 15. The method of any of the preceding Clauses, further comprising at least one of: providing a recorded guide in a 2D or 3D format to deliver and guide the user within an XR environment, wherein the first user input corresponds to one of a plurality of input modalities for user interaction, and is received via one of: an analog stick, a pointer, gaze tracking, hand pointing, and hand gestures; sending a prescription to an online retail account or an email address associated with the user; and creating a virtual environment for a vision test that simulates an expansive space with audio cues for user guidance and feedback.

Clause 16. The method of any of the preceding Clauses, further comprising: dynamically adjusting the first set of optotypes or the second set of optotypes based on user performance, including enabling an adaptive focus effect; and wherein displaying the first set of optotypes and displaying the second set of optotypes includes applying overlay rendering to simulate physics of XR lenses to reduce distortion in the first set of optotypes and the second set of optotypes.

Clause 17. The method of any of the preceding Clauses, further comprising: executing a vision test game including a dynamic content presentation and interactive user experiences; measuring and calibrating an interpupillary distance (IPD) within one or more XR settings to tailor examination to individual user specifications.

Clause 18. The method of any of the preceding Clauses, further comprising: determining the user's physical distance from the display; determining the visual acuity based on the user's physical distance from the display; and adjusting the assessed visual acuity of the user based on user movement within a virtual XR environment.

Clause 19. The method of any of the preceding Clauses, further comprising: providing a virtual phoropter within an associated XR environment, and enabling simulation of lens adjustments and refractive error measurements through virtual interaction.

Clause 20. The method of any of the preceding Clauses, displaying the first set of optotypes further comprising: rotating the first set of optotypes on the display; and varying a size of the first set of optotypes.

Clause 21. The method of any of the preceding Clauses, further comprising: creating a virtual immersive testing environment including a Zen modern office, wherein the first set of optotypes and the second set of optotypes are displayed in the virtual immersive testing environment.

Clause 22. The method of any of the preceding Clauses, further comprising: determining a level of visual perception of the user using the XR device, including: applying visual stimuli including a series of light-based patterns or gradients that gradually transition in intensity or color; receiving a series of user inputs identifying perceived changes or thresholds in response to the visual stimuli; and based on the series of user inputs, determining the level of visual perception indicating a sensitivity to contrast or color gradients.

Clause 23. The method of any of the preceding Clauses, further comprising: while displaying the first set of optotypes, creating a plurality of lighting conditions within an associated XR environment to mimic real-world visual scenarios and assess the visual acuity of the user under a plurality of ambient light settings corresponding to the plurality of lighting conditions.

Clause 24. The method of any of the preceding Clauses, wherein the XR system further includes: applying, by a lens simulation module, color and rendering distortions to the first set of optotypes or the second set of optotypes based on an optical model.

Clause 25. The method of any of the preceding Clauses, further comprising: creating an XR environment including displaying a continuous control interface in the XR environment, the first set of optotypes and the second set of optotypes displayed on the continuous control interface; in response to the first user input, assessing and adjusting a cylinder power and an axis; and determining astigmatism correction based on the cylinder power and the axis.

Clause 26. The method of any of the preceding Clauses, further comprising: dynamically adjusting a perceived distance of the first set of optotypes or the second set of optotypes from the user.

Clause 27. The method of any of the preceding Clauses, further comprising: providing, by an audio feedback mechanism, verbal cues or sounds in response to the first user input, enhancing interactive experience and guiding the user through a testing process.

Clause 28. The method of any of the preceding Clauses, further comprising: in accordance with a virtual character-guided testing feature, displaying a character selected from: realistic human figures, cartoon characters, and abstract forms.

Clause 29. The method of any of the preceding Clauses, further comprising: recognizing a hand gesture of the user by a hand gesture recognition system; determining the first user input based on the hand gesture in response to displaying the first set of optotypes.

Clause 30. The method of any of the preceding Clauses, further comprising: displaying a user interface on the display for selecting optotypes or responding to a test prompt, wherein the user interface is configured to be displayed adaptively with a plurality of formats for users having different abilities and preferences.

Clause 31. The method of any of the preceding Clauses, comprising: storing test results in a database storing external health records or in a vision care provider system, facilitating a transfer of the test results for further clinical review or analysis.

Clause 32. The method of any of the preceding Clauses, further comprising: generating a report including one or more of: the user's visual acuity, perception levels, and potential corrective lens prescriptions, the report having a standard format that are applied among eye care professionals or patients.

Clause 33. A non-transitory computer readable storage medium, storing one or more programs for execution by one or more processors of a computing device associated with a plurality of electronic devices in a smart home environment, the plurality of electronic devices together composing the smart home environment at least in part, and the smart home environment having a plurality of environment statuses, the one or more programs including instructions for performing a method in any of Clauses 1-32.

Clause 34. An extended reality (XR) device, comprising: one or more processors; a display, a microphone, a speaker, and one or more sensors including at least an eye tracking device; memory for storing one or more programs for execution by the one or more processors, the one or more programs including instructions for performing a method in any of Clauses 1-32.

Clause 35. A virtual reality (VR) system for vision testing, comprising: means for displaying optotypes overcoming pixel density limitations of VR displays through algorithmic enhancement; a calibration system for aligning a user's foveal vision with the VR display's central axis using eye-tracking technology; voice control functionality enabling users to navigate and respond within the VR vision testing protocol through spoken commands; a virtual representation of an optometrist placed within the VR environment for guiding the user through the vision test; optotypes including but not limited to tumbling E and tumbling C, with adaptive sizing and orientation based on user performance; a continuous search method implemented for determining visual acuity with precision beyond standard categorization; a forced choice method for acuity and sphere determination, offering minimal selection options to refine prescription accuracy; an algorithm for optotype selection incorporating a confidence scoring mechanism to verify visual prescription before finalization; a method for simulating fixed-distance optotype presentation adjusted according to user's position and viewpoint within the VR environment; functionality to occlude vision of one eye at a time within the VR headset for isolated eye testing.

Clause 36. The VR system of Clause 35, further comprising: a recorded guide in 2D or 3D format to deliver and guide the vision test within the VR environment; multiple input modalities for user interaction during the test, including but not limited to analog stick, pointer, gaze tracking, hand pointing, and hand gestures; a method for delivering prescriptions directly to users, including association with an online retail account or emailing from the VR environment; a virtual environment for the vision test that simulates an expansive space with audio cues for user guidance and feedback.

Clause 37. The VR system of any of Clauses 35 to 36, wherein the system is configured to: dynamically adjust the display of optotypes based on an algorithm that accounts for user performance, allowing adaptive focus on specific visual challenges; employ a unique overlay rendering technique that simulates the physics of VR lenses to accurately display optotypes, addressing potential distortions.

Clause 38. The VR system of any of Clauses 35 to 37, wherein the system utilizes: game engine technologies for executing the vision tests, leveraging dynamic content presentation and interactive user experiences; a method for measuring and calibrating interpupillary distance (IPD) within the VR setting to tailor the examination to individual user specifications.

Clause 39. The VR system of any of Clauses 35 to 38, further comprising: a mechanism for recalculating visual acuity based on the user's physical distance from optotype displays, employing an algorithm to adjust acuity values according to user movement within the VR environment.

Clause 40. The VR system of any of Clauses 35 to 39, further configured to provide a virtual phoropter within the VR environment, enabling the simulation of lens adjustments and refractive error measurements through virtual interaction.

Clause 41. The VR system of any of Clauses 35 to 40, wherein the adaptive optotype presentation system includes a method for arbitrarily rotating and sizing optotypes to test visual acuity under varied spatial orientations and scales.

Clause 42. The VR system of any of Clauses 35 to 41, further comprising: a comfortable and immersive testing environment designed to mimic a zen modern office, aimed at relaxing the user to minimize error rates in visual acuity testing.

Clause 43. The VR system of any of Clauses 35 to 42, wherein the system is further capable of: designing and testing new unique optotypes, enabling the exploration of alternative visual acuity and perception testing methods beyond traditional characters and symbols.

Clause 44. The VR system of any of Clauses 35 to 43, further comprising: a method for determining the level of visual perception of a person in the VR headset without needing to distinguish traditional optotypes, utilizing a series of light-based patterns or gradients that gradually transition in intensity or color, requiring the user to identify changes or thresholds in the visual stimulus, thus assessing visual perception based on sensitivity to contrast or color gradients rather than shape recognition.

Clause 45. The VR system of any of Clauses 35 to 44, additionally configured to: simulate various lighting conditions within the VR environment to assess the user's visual acuity and perception under different ambient light settings, mimicking real-world visual scenarios.

Clause 46. The VR system of any of Clauses 35 to 45, wherein the system includes: a lens simulation feature that applies color and rendering distortions based on mathematical models of optical physics, to accurately replicate the effects of various lens prescriptions on perceived optotypes.

Clause 47. The VR system of any of Clauses 35 to 46, further comprising: a continuous control interface within the VR environment for determining astigmatism correction by adjusting and assessing cylinder power and axis through user interaction.

Clause 48. The VR system of any of Clauses 35 to 47, wherein the system employs: a method for dynamically adjusting the perceived distance of optotypes from the user, simulating closer or farther visual tasks without the user physically moving within the VR environment.

Clause 49. The VR system of any of Clauses 35 to 48, further comprising: an audio feedback mechanism that provides verbal cues or sounds in response to user actions during the test, enhancing the interactive experience and guiding the user through the testing process.

Clause 50. The VR system of any of Clauses 35 to 49, wherein the virtual character-guided testing is configured to provide multiple character options, including realistic human figures, cartoon characters, and abstract forms, to cater to user preferences and enhance engagement.

Clause 51. The VR system of any of Clauses 35 to 50, further configured to incorporate: a hand gesture recognition system for interacting with the VR vision test, allowing users to select responses or navigate the testing protocol through natural movements.

Clause 52. The VR system of any of Clauses 35 to 51, wherein the system features: multiple redundant user interface modalities for selecting optotypes or responding to tests, ensuring accessibility for users with varying abilities and preferences.

Clause 53. The VR system of any of Clauses 35 to 52, further comprising: a method for integrating test results with external health records or vision care provider systems, facilitating the seamless transfer of data for clinical review or further analysis.

Clause 54. The VR system of any of Clauses 35 to 53, wherein the system is capable of generating: a comprehensive report detailing the user's visual acuity, perception levels, and potential corrective lens prescriptions, formatted for easy interpretation by eye care professionals or the user themselves.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

As used herein, the word “module” refers to logic embodied in hardware or firmware, or to a collection of software instructions, possibly having entry and exit points, written in a programming language, such as, for example C++. A software module may be compiled and linked into an executable program, installed in a dynamic link library, or may be written in an interpretive language such as BASIC. It will be appreciated that software modules may be callable from other modules or from themselves, and/or may be invoked in response to detected events or interrupts. Software instructions may be embedded in firmware, such as an EPROM or EEPROM. It will be further appreciated that hardware modules may be comprised of connected logic units, such as gates and flip-flops, and/or may be comprised of programmable units, such as programmable gate arrays or processors. The modules described herein are preferably implemented as software modules, but may be represented in hardware or firmware.

It is contemplated that the modules may be integrated into a fewer number of modules. One module may also be separated into multiple modules. The described modules may be implemented as hardware, software, firmware or any combination thereof. Additionally, the described modules may reside at different locations connected through a wired or wireless network, or the Internet.

In general, it will be appreciated that the processors can include, by way of example, computers, program logic, or other substrate configurations representing data and instructions, which operate as described herein. In other embodiments, the processors can include controller circuitry, processor circuitry, processors, general purpose single-chip or multi-chip microprocessors, digital signal processors, embedded microprocessors, microcontrollers and the like.

Furthermore, it will be appreciated that in one embodiment, the program logic may advantageously be implemented as one or more components. The components may advantageously be configured to execute on one or more processors. The components include, but are not limited to, software or hardware components, modules such as software modules, object-oriented software components, class components and task components, processes methods, functions, attributes, procedures, subroutines, segments of program code, drivers, firmware, microcode, circuitry, data, databases, data structures, tables, arrays, and variables.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Furthermore, to the extent that the term “include,” “have,” or the like is used in the description or the claims, such term is intended to be inclusive in a manner similar to the term “comprise” as “comprise” is interpreted when employed as a transitional word in a claim.

As used herein, the term “about” is relative to the actual value stated, as will be appreciated by those of skill in the art, and allows for approximations, inaccuracies and limits of measurement under the relevant circumstances. In one or more aspects, the terms “about,” “substantially,” and “approximately” may provide an industry-accepted tolerance for their corresponding terms and/or relativity between items, such as a tolerance of from less than one percent to 10% percent of the actual value stated, and other suitable tolerances.

As used herein, the term “comprising” indicates the presence of the specified integer(s), but allows for the possibility of other integers, unspecified. This term does not imply any particular proportion of the specified integers. Variations of the word “comprising,” such as “comprise” and “comprises,” have correspondingly similar meanings.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

Although the detailed description contains many specifics, these should not be construed as limiting the scope of the subject technology but merely as illustrating different examples and aspects of the subject technology. It should be appreciated that the scope of the subject technology includes other embodiments not discussed in detail above. Various other modifications, changes and variations may be made in the arrangement, operation and details of the method and apparatus of the subject technology disclosed herein without departing from the scope of the present disclosure. In addition, it is not necessary for a device or method to address every problem that is solvable (or possess every advantage that is achievable) by different embodiments of the disclosure in order to be encompassed within the scope of the disclosure. The use herein of “can” and derivatives thereof shall be understood in the sense of “possibly” or “optionally” as opposed to an affirmative capability.

What is claimed:

1. A method for assessing visual acuity, comprising: at an extended reality (XR) device including one or more processors, memory for storing one or more programs, a display, a microphone, a speaker, and one or more sensors including at least an eye tracking device:
identifying a nominal foveal location of an eye using the eye tracking device;
setting a central axis of the display based on the nominal foveal location;
based on the central axis of the display, displaying a first set of optotypes on the display, the first set of optotypes including one or more first optotypes;
obtaining a first user input in response to displaying the first set of optotypes;
in response to the first user input, determining a size progression from the first set of optotypes to a second set of optotypes, the first set of optotypes including one or more second optotypes and
displaying the second set of optotypes on the display based on the size progression for assessing visual acuity of a user.

2. The method of claim 1, wherein the first set of optotypes and the second set of optotypes are displayed concurrently on the display.

3. The method of claim 1, wherein the first set of optotypes and the second set of optotypes are displayed sequentially via two successive video clips or two static images on the display, and the size progression is determined and the second set of optotypes are displayed adaptively based on the first user input.

4. The method of claim 1, wherein the size progression corresponds to a visual acuity assessment resolution that is different from any known visual acuity assessment resolution of a plurality of predefined eye charts.

5. The method of claim 1, further comprising based on an optotype size of the first set of optotypes, an optotype size of the second set of optotypes, the first user input, and the size progression, determining a visual prescription.

6. The method of claim 1, further comprising:
determining a confidence score for determining the size progress of the second set of optotypes; and
verifying a visual prescription based on the confidence score.

7. The method of claim 1, wherein the display has a pixel resolution, and the first set and the second set of optotypes are displayed with a second resolution that is greater than the pixel resolution.

8. The method of claim 1, wherein the display includes a left display and a right display, and the first set and the second set of optotypes are displayed in one of the left display and the right display, the method further comprising displaying a static blank image in the other one of the left display and the right display to occlude vision of a corresponding eye.

9. The method of claim 1, further comprising:
providing a virtual phoropter within an associated XR environment, and
enabling simulation of lens adjustments and refractive error measurements through virtual interaction.

10. The method of claim 1, displaying the first set of optotypes further comprising:
rotating the first set of optotypes on the display; and
varying a size of the first set of optotypes.

11. The method of claim 1, further comprising:
creating a virtual immersive testing environment including a Zen modern office, wherein the first set of optotypes and the second set of optotypes are displayed in the virtual immersive testing environment.

12. The method of claim 1, further comprising:
determining a level of visual perception of the user using the XR device, including:
applying visual stimuli including a series of light-based patterns or gradients that gradually transition in intensity or color;
receiving a series of user inputs identifying perceived changes or thresholds in response to the visual stimuli; and
based on the series of user inputs, determining the level of visual perception indicating a sensitivity to contrast or color gradients.

13. The method of claim 1, further comprising, while displaying the first set of optotypes, creating a plurality of lighting conditions within an associated XR environment to mimic real-world visual scenarios and assess the visual acuity of the user under a plurality of ambient light settings corresponding to the plurality of lighting conditions.

14. The method of claim 1, further comprising:
creating an XR environment including displaying a continuous control interface in the XR environment, the first set of optotypes and the second set of optotypes displayed on the continuous control interface;
in response to the first user input, assessing and adjusting a cylinder power and an axis; and
determining astigmatism correction based on the cylinder power and the axis.

15. An extended reality (XR) device, comprising:
one or more processors;
a display, a microphone, a speaker, and one or more sensors including at least an eye tracking device;
memory for storing one or more programs for execution by the one or more processors, the one or more programs including instructions for performing the method of claim 1.

16. A virtual reality (VR) system for vision testing, comprising:
means for displaying optotypes overcoming pixel density limitations of VR displays through algorithmic enhancement;
a calibration system for aligning a user's foveal vision with the VR display's central axis using eye-tracking technology;
voice control functionality enabling users to navigate and respond within the VR vision testing protocol through spoken commands;
a virtual representation of an optometrist placed within the VR environment for guiding the user through the vision test;
optotypes including but not limited to tumbling E and tumbling C, with adaptive sizing and orientation based on user performance;
a continuous search method implemented for determining visual acuity with precision beyond standard categorization;
a forced choice method for acuity and sphere determination, offering minimal selection options to refine prescription accuracy; an algorithm for optotype selection incorporating a confidence scoring mechanism to verify visual prescription before finalization;
a method for simulating fixed-distance optotype presentation adjusted according to user's position and viewpoint within the VR environment;
functionality to occlude vision of one eye at a time within the VR headset for isolated eye testing.

17. The VR system of claim 16, further comprising:
a recorded guide in 2D or 3D format to deliver and guide the vision test within the VR environment;
multiple input modalities for user interaction during the test, including but not limited to analog stick, pointer, gaze tracking, hand pointing, and hand gestures;
a method for delivering prescriptions directly to users, including association with an online retail account or emailing from the VR environment;
a virtual environment for the vision test that simulates an expansive space with audio cues for user guidance and feedback.

18. The VR system of claim 16, wherein the system is configured to:
dynamically adjust the display of optotypes based on an algorithm that accounts for user performance, allowing adaptive focus on specific visual challenges;
employ a unique overlay rendering technique that simulates the physics of VR lenses to accurately display optotypes, addressing potential distortions.

19. The VR system of claim 16, wherein the system utilizes:
game engine technologies for executing the vision tests, leveraging dynamic content presentation and interactive user experiences;
a method for measuring and calibrating interpupillary distance (IPD) within the VR setting to tailor the examination to individual user specifications.

20. The VR system of claim 16, wherein the system includes a lens simulation feature that applies color and rendering distortions based on mathematical models of optical physics, to accurately replicate the effects of various lens prescriptions on perceived optotypes.
